(19) United States
(12) Patent Application Publication
(10) Pub. No.: US 2025/0127106 A1
(43) Pub. Date: Apr. 24, 2025

(54) PERONOSPORA RESISTANCE IN SPINACIA OLERACEA

(71) Applicant: RIJK ZWAAN ZAADTEELT EN ZAADHANDEL B.V., De Lier (NL)

(72) Inventors: Vincent Laurens Adrianus KOCK, De Lier (NL); Raoul Jacobus Johannes Maria FRIJTERS, De Lier (NL)

(21) Appl. No.: 18/923,953

(22) Filed: Oct. 23, 2024

Related U.S. Application Data
(63) Continuation-in-part of application No. PCT/EP2023/059918, filed on Apr. 17, 2023.

(30) Foreign Application Priority Data
Apr. 28, 2022 (WO) PCT/EP2022/061377

(51) Int. Cl. A01H 1/00; A01H 5/12; A01H 6/02; C07K 14/415; C12Q 1/6895

(52) U.S. Cl. CPC A01H 1/1255; A01H 5/12; A01H 6/028; C07K 14/415; C12Q 1/6895; C12Q 2600/13; C12Q 2600/156

(57) ABSTRACT

The present invention relates to an allele designated alpha-WOLF 23 which confers resistance to at least one Peronospora effusa race, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the protein has an amino acid sequence that in order of increased preference has at least 93.5%, 94%, 94.5%, 95%, 95.5%, 96%, 96.5%, 97%, 97.5%, 98%, 98.5%, 99%, 99.5%, 100% sequence similarity to SEQ ID NO: 11. The allele when present in a spinach plant confers complete resistance to at least Peronospora effusa races Pe:3, Pfs:5, Pe:8, Pe:9, Pe:11, Pe:14 and Pe:16 and does not confer resistance to races Pe:2, Pe:4, Pe:6, Pe:7, Pe:10, Pe:13, Pe:15 and Pe:18.

### RELATED APPLICATIONS AND INCORPORATION BY REFERENCE

This application is a continuation-in-part application of international patent application Serial No. PCT/EP2023/059918 filed Apr. 17, 2023, which published as PCT Publication No. WO 2023/208632 on Nov. 2, 2024, which claims benefit of international patent application Serial No. PCT/EP2022/061377 filed Apr. 28, 2022.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created Oct. 16, 2024, is named Y7954_00641SL.xml and is 27,002 bytes in size.

### FIELD OF THE INVENTION

The invention relates to an allele conferring resistance against downy mildew (Peronospora effusa), and plants comprising said allele. The invention further relates to progeny, seed, and plant parts such as leaves of said resistant plant; and the invention relates to propagation material suitable for producing said plant. The invention also relates to methods for identifying the allele and resistant plant, and to methods for selecting and producing the resistant plant.

### BACKGROUND OF THE INVENTION

Downy mildew (Peronospora effusa) is a major threat for spinach growers because it directly affects the harvested leaves. In spinach, downy mildew is caused by the oomycete Peronospora effusa (formerly known as Peronospora farinose f. sp. spinaciae). Infection makes the leaves unsuitable for sale and consumption, as it manifests itself phenotypically as yellow lesions on the older leaves, and on the abaxial leaf surface a greyish fungal growth can be observed. The infection can spread very rapidly, and it can occur both in glasshouse cultivation and in soil cultivation. The optimal temperature for formation and germination of P. effusa spores is 9 to 12° C., and it is facilitated by a high relative humidity. When spores are deposited on a humid leaf surface they can readily germinate and infect the leaf. Fungal growth is optimal between 8 and 20° C. and a relative humidity of >80%, and within 6 and 13 days after infection mycelium growth can be observed. Oospores of P. effusa can survive in the soil for up to 3 years, or as mycelium in seeds or living plants.

To date 19 pathogenic races of spinach downy mildew (Pe, formerly known as Pfs) have been officially identified and characterized, and many new candidates are observed in the field. The 19 officially recognized races of Peronospora effusa, are designated Pe:1 to Pe:19 (Pe:1 to Pe:17 were formerly known as Pfs:1 to Pfs17 (Irish et al. Phtypathol. Vol. 98 pg. 894-900, 2008; Plantum NL (Dutch association for breeding, tissue culture, production and trade of seed and young plants) press release, “Benoeming van Pfs: 14, een nieuwe fysio van valse meeldauw in spinazie”, Sep. 19, 2012; Report Jim Correl (Univ. Arkansas) and Steven Koike (UC Cooperative Extension, Monterey County), “Race Pfs: 14—Another new race of the spinach downy mildew pathogen”, Sep. 18, 2012; Plantum NL press release, “Denomination of Pfs: 15, a new race of downy mildew in spinach”, Sep. 2, 2014; Plantum NL press release, “Denomination of Pfs: 16, a new race of downy mildew in spinach, Mar. 15, 2016; Plantum NL press release, Denomination of Pfs: 17, a new race of downy mildew in spinach”, Apr. 16, 2018; Plantum NL press release). Pe: 18 and 19 are described in “Denomination of Pe: 18 and 19, two new races of downy mildew in spinach”, Apr. 15, 2021)).

All 19 officially recognized Pe races are publicly available from Naktuinbouw, Sotaweg 22, 2371 GD Roelofarendsveen, the Netherlands.

Especially the latest identified Peronospora races can break the resistance of many spinach varieties that are currently used commercially worldwide, and they thus pose a serious threat to the productivity of the spinach industry. Therefore, it is crucial to stay at the forefront of developments in this field, as Peronospora continuously develops the ability to break the resistances that are present in commercial spinach varieties. For this reason, new resistance genes against downy mildew are very valuable assets, and they form an important research focus in breeding and particular in spinach and lettuce breeding. One of the main goals of spinach breeders is to rapidly develop spinach varieties with a resistance to as many Peronospora races as possible, including the latest identified races, before these races become wide-spread and pose a threat to the industry.

In commercial spinach varieties, resistance against downy mildew is usually caused by so-called R-genes. R-gene mediated resistance is based on the ability of a plant to recognize the invading pathogen. In many cases this recognition occurs after the pathogen has established the first phases of interaction and transferred a so called pathogenicity (or avirulence) factor into the plant cell. These pathogenicity factors interact with host components in order to establish conditions which are favorable for the pathogen to invade the host and thereby cause disease. When a plant is able to recognize the events triggered by the pathogenicity factors a resistance response can be initiated. In many different plant pathogen interaction systems such as the interaction of spinach with different downy mildew strains, the plant initiates these events only after specific recognition of the invading pathogen.

Co-evolution of plant and pathogen has led to an arms race in which a R-gene mediated resistance is sometimes overcome as a consequence of the capability of the pathogen to interact with and modify alternative host targets or the same targets in a different way, such that the recognition is lost and infection can be established successfully resulting in disease. In order to re-establish resistance in a plant, a new R-gene has to be introduced which is able to recognize the mode of action of an alternative pathogenicity factor.

Despite the fact that the durability of R-genes is relatively low, R-genes are in spinach still the predominant form of defense against downy mildew. This is mainly due to the fact that it is the only form of defense that gives absolute resistance. So far plant breeders have been very successful in generating downy mildew resistant spinach varieties by making use of resistance genes residing in the wild germplasm of the crop species. Even though R-genes are extensively used in spinach breeding, until now not much is known of these R-genes.

Citation or identification of any document in this application is not an admission that such document is available as prior art to the present invention.

### SUMMARY OF THE INVENTION

The R-genes officially recognized in spinach are in fact all different alleles of the two tightly linked genes, the alpha- and the beta-WOLF genes (WO2018/060474; Kock et al.). This was also the first time that R-genes, or better R-alleles, were characterized at the molecular level, i.e. their nucleotide and amino acid sequence was determined. Although this provides the breeder with tools that increase the efficiency of detecting and selecting R-alleles, adequately responding to newly emerging downy mildew races is still crucial for developing commercially successful spinach varieties.

In the research leading to the present invention, a new allelic variant of the Alpha-WOLF gene as described in WO2018059651 was found. The alpha-WOLF gene encodes a protein that belongs to the CC-NBS-LRR family (Coiled Coil-Nucleotide Binding Site-Leucine-Rich Repeat).

In the context of this invention the term “allele” or “allelic variant” is used to designate a version of the gene that is linked to a specific phenotype, i.e. resistance profile. It was found that a spinach plant may carry one or two WOLF genes. Each of these two WOLF genes encompasses multiple alleles, each allele conferring a particular resistance profile.

The beta WOLF gene is located on scaffold12735 (sequence: GenBank: KQ143339.1), at position 213573-221884. In case the spinach plant also carries or only carries the alpha-WOLF gene, the alpha-WOLF gene is located at approximately the same location as where the beta-WOLF gene is located on scaffold12735 in the Viroflay genome assembly.

The present invention provides for an allele of an alpha-WOLF gene encoding a protein which confers at least resistance to Peronospora effusa races Pe:3, Pfs:5, Pe:8, Pe:9, Pe:11, Pe:14 and Pe:16 and does not confer resistance to races Pe:2, Pe:4, Pe:6, Pe:7, Pe:10, Pe:13, Pe:15 and Pe:18 when expressed in a spinach plant, and wherein the protein comprises in its amino acid sequence the motif “MAEIGYSVC” (SEQ ID NO: 1) at its N-terminus, and the motif “KWMCLR” (SEQ ID NO: 2); and wherein said allele comprises:
a) a nucleotide sequence comprising a coding sequence that has at least 97% sequence identity to SEQ ID NO: 10, or
b) a nucleotide sequence encoding a protein which has an amino acid sequence that has at least 93.5% sequence similarity to SEQ ID NO: 11, or
c) a nucleotide sequence encoding an LRR domain which nucleotide sequence has at least 94.5% sequence identity to SEQ ID NO: 12, or
d) a nucleotide sequence encoding an LRR domain which has an amino acid sequence that has at least 93% sequence similarity to SEQ ID NO: 13.

In some embodiments, the protein encoded by the allele further comprises the motif “DQEDEGEDN” (SEQ ID NO: 4).

In some embodiments, the allele confers complete resistance to at least Pe:3, Pfs:5, Pe:9, Pe:11, and Pe:14 when homozygously present in a spinach plant.

In some embodiments, the present invention relates to protein encoded by the allele of the alpha-WOLF gene, which confers at least resistance to Peronospora effusa races Pe:3, Pfs:5, Pe:8, Pe:9, Pe:11, Pe:14 and Pe:16 and does not confer resistance to races Pe:2, Pe:4, Pe:6, Pe:7, Pe:10, Pe:13, Pe:15 and Pe:18 when expressed in a spinach plant, and wherein the protein comprises in its amino acid sequence the motif “MAEIGYSVC” (SEQ ID NO: 1) at its N-terminus, and the motif “KWMCLR” (SEQ ID NO: 2).

In some embodiments, the present invention relates to a spinach plant comprising the allele of the alpha-WOLF gene.

In some embodiments, the plant is an agronomically elite plant, in particular a hybrid variety or an inbred line.

In some embodiments the present invention relates to a spinach seed comprising the allele of the alpha-WOLF gene, which is capable of growing into a spinach plant.

In some embodiments the present invention relates to propagation material suitable for producing the spinach plant as described herein as claimed in any one of the claims 5 and 6, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the allele of the alpha-WOLF gene.

According to another aspect of the present invention, the invention further provides for a method for identifying a spinach plant comprising the allele as described herein, wherein the method comprises the following steps:
a) detecting the allele as described herein in the genome of a plant by determining the sequence of the allele, or
b) detecting the sequence of the LRR domain of the allele as described herein in the genome of a plant, or
c) by detecting a unique polymorphism in the allele as described herein, and optionally
d) testing of the plant comprising the allele of the invention for exhibiting resistance to Peronospora effusa.

In some embodiments, the LRR domain is determined by using a primer pair to amplify the LRR domain, wherein the forward primer is a nucleic acid molecule having the sequence of SEQ ID NO: 5.

In some embodiments, the LRR domain is determined by using a primer pair to amplify the LRR domain, wherein the reverse primer is a nucleic acid molecule having the sequence of SEQ ID NO: 6.

In some embodiments, the present invention provides for a method for selecting a spinach plant resistant to Peronospora effusa, comprising identifying the presence of the allele of the alpha-WOLF gene and optionally testing the plant for resistance against Peronospora effusa, and selecting a plant comprising said allele as a plant which is resistant to at least Peronospora effusa races Pe:3, Pfs:5, Pe:9, Pe:11, and Pe:14, preferably to Pe:3, Pfs:5, Pe:8, Pe:9, Pe:11, Pe:14 and Pe:16.

In some embodiments, the present invention provides for a method for producing a spinach plant resistant to Peronospora effusa races Pe:3, Pfs:5, Pe:8, Pe:9, Pe:11, Pe:14 and Pe:16 comprising the steps of:
a) crossing a first parent plant comprising the allele of the alpha-WOLF gene with a second parent plant to obtain an F1 population;
b) optionally performing one or more rounds of selfing and/or crossing with a plant from the F1 population to obtain a further generation population;
c) selecting from the F1 population or the further generation population a plant that comprises the allele of the alpha-WOLF gene as a resistant plant.

In some embodiments, the present invention provides for a method for producing hybrid spinach seed resistant to Peronospora effusa, comprising the steps of crossing a first parent plant with a second parent plant, wherein one or both parent plants are homozygous for the allele of the alpha-WOLF gene and harvesting the hybrid seed. In some embodiments, the present invention provides for a hybrid seed produced by the method. In some embodiments, the present invention provides for a plant grown from the hybrid seed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION OF THE INVENTION

It is the object of the invention to provide a new resistance allele of the alpha-WOLF gene and to provide molecular biological tools for identifying this new resistance allele. The variation observed between alleles of the alpha- and beta-WOLF genes that have been identified is enormous and not straightforward. Therefore, the resistance pattern that is conferred by a WOLF-allele cannot be predicted beforehand.

A genome assembly for spinach variety Viroflay—which is susceptible to all known pathogenic races of Peronospora effusa—is publicly available (Spinacia oleracea cultivar SynViroflay, whole genome shotgun sequencing project; Bioproject: PRJNA41497; GenBank: AYZV00000000.2; BioSample: SAMN02182572, see also Dohm et al, 2014, Nature 505: 546-549). In this genome assembly for Viroflay, the beta-WOLF gene is located on scaffold12735 (sequence: GenBank: KQ143339.1), at position 213573-221884. The sequence covered by this interval comprises the entire genomic sequence of the beta-WOLF gene of Viroflay, plus 2000 basepairs sequence upstream from the gene, plus the sequence downstream from the gene, up to the locus of the neighbouring gene that is situated downstream from the WOLF gene. Spinach variety Viroflay only possesses a single WOLF gene, namely a beta-WOLF gene, but most other spinach lines harbor a single alpha-type WOLF gene at the same location in the genome. Other spinach lines harbor two WOLF genes at approximately the same location in the genome. In such cases, the two WOLF genes are positioned adjacent to each other. In most spinach lines that harbor two WOLF genes, one of said WOLF genes belongs to the alpha-type, and the other WOLF gene belongs to the beta-type. It was observed that this allelic variation in the WOLF locus is responsible for differences in resistance to pathogenic races of Peronospora effusa.

The difference between an allele of an alpha-WOLF gene and an allele of a beta-WOLF gene lies in the presence of specific conserved amino acid motifs in the encoded protein sequence. As mentioned above, all WOLF proteins possess—from N- to C-terminus—the following domains that are generally known in the art: a coiled coil domain (RX-CC-like, cd14798), an NBS domain (also referred to as “NB-ARC domain”, pfam00931; van der Biezen & Jones, 1998, Curr. Biol. 8: R226-R228), and leucine-rich repeats (IPR032675) which encompass the LRR domain. In addition, all WOLF proteins comprise in their amino acid sequence the motif “MAEIGYSVC” (SEQ ID NO: 1) at the N-terminus. In addition to this, all alpha-WOLF proteins comprise the motif “KWMCLR” (SEQ ID NO: 2) in their amino acid sequence, whereas all beta-WOLF proteins comprise the motif “HVGCVVDR” (SEQ ID NO: 3) in their amino acid sequence.

The present invention relates to a new downy mildew resistance conferring allele of the alpha-WOLF gene, herein referred to as the ‘allele of the invention’ or ‘alpha-WOLF 23 allele’, encoding a protein which confers resistance to Peronospora effusa when expressed in a spinach plant.

The alpha-WOLF 23 allele encodes a CC-NBS-LRR protein which confers resistance to Peronospora effusa races Pe:3, Pfs:5, Pe:8, Pe:9, Pe:11, Pe:14 and Pe:16 when expressed in a spinach plant, and wherein the protein comprises in its amino acid sequence the motif “MAEIGYSVC” (SEQ ID NO: 1) at its N-terminus, and the motif “KWMCLR” (SEQ ID NO: 2); and wherein said allele comprises:
a) a nucleotide sequence comprising a coding sequence that in order in order of increased preference has at least 97%, 97.5%, 98%, 98.5%, 99%, 99.5%, 100% sequence identity to SEQ ID NO: 10, or
b) a nucleotide sequence encoding a protein which has an amino acid sequence that in order of increased preference has at least 93.5%, 94%, 94.5%, 95%, 95.5%, 96%, 96.5%, 97%, 97.5%, 98%, 98.5%, 99%, 99.5%, 100% sequence similarity to SEQ ID NO: 11, or
c) a nucleotide sequence encoding an LRR domain which nucleotide sequence in order of increased preference has at least 94.5%, 95%, 95.5%, 96%, 96.5%, 97%, 97.5%, 98%, 98.5%, 99%, 99.5%, 100% sequence identity to SEQ ID NO: 12, or
d) a nucleotide sequence encoding an LRR domain which has an amino acid sequence that in order of increased preference has at least 93%, 93.5%, 94.5%, 95%, 95.5%, 96%, 96.5%, 97%, 97.5%, 98%, 98.5%, 99%, 99.5%, 100% sequence similarity to SEQ ID NO: 13.

Optionally, the protein encoded by the alpha-WOLF 23 allele further comprises an additional motif in its amino acid sequence, namely “DQEDEGEDN” (SEQ ID NO: 4).

The allele of the invention is a nucleic acid, in particular a nucleic acid molecule, more in particular an isolated nucleic acid molecule.

The allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a coding sequence that in order in order of increased preference has at least 97%, 97.5%, 98%, 98.5%, 99%, 99.5%, 100%97%, 97.5%, 98%, 98.5%, 99%, 99.5%, 100% sequence identity to SEQ ID NO: 10.

Preferably, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a coding sequence that in order in order of increased preference has at least 97% sequence identity to SEQ ID NO: 10.

In a preferred embodiment, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a coding sequence according to SEQ ID NO: 10.

The allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding a protein which has an amino acid sequence that has at least 93.5% sequence similarity to SEQ ID NO: 11. Preferably, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding a protein which has an amino acid sequence that in order of increased preference has at least 94%, 94.5%, 95%, 95.5%, 96%, 96.5%, 97%, 97.5%, 98%, 98.5%, 99%, 99.5%, 100% sequence similarity to SEQ ID NO: 11.

In a preferred embodiment, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding a protein having an amino acid sequence according to SEQ ID NO: 11.

The allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding an LRR domain which nucleotide sequence in order of increased preference has at least 94.5% sequence identity to SEQ ID NO: 12.

Preferably, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding an LRR domain which nucleotide sequence in order of increased preference has at least 95%, 95.5%, 96%, 96.5%, 97%, 97.5%, 98%, 98.5%, 99%, 99.5%, 100% sequence identity to SEQ ID NO: 12.

In a preferred embodiment, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding an LRR domain which has a nucleotide sequence according to SEQ ID NO: 12.

The allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding an LRR domain which has an amino acid sequence that has at least 93% sequence similarity to SEQ ID NO: 13.

Preferably, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding an LRR domain which has an amino acid sequence that in order of increased preference has at least 93.5%, 94.5%, 95%, 95.5%, 96%, 96.5%, 97%, 97.5%, 98%, 98.5%, 99%, 99.5%, 100% sequence similarity to SEQ ID NO: 13.

In a preferred embodiment, the allele of the invention encodes a CC-NBS-LRR protein, wherein the protein comprises in its amino acid sequence the motif “MAEIGYSVC” (SEQ ID NO: 1) at its N-terminus, and the motif “KWMCLR” (SEQ ID NO: 2), and wherein the allele comprises a nucleotide sequence encoding an LRR domain which has an amino acid sequence according to SEQ ID NO: 13.

The alpha-WOLF 23 allele when homozygously present in a spinach plant confers complete resistance to at least Peronospora effusa races Pe:3, Pfs:5, Pe:8, Pe:9, Pe:11, Pe:14 and Pe:16, and does not confer resistance to at least Peronospora effusa races Pe:2, Pe:4, Pe:6, Pe:7, Pe:10, Pe:13, Pe:15 and Pe:18.

The alpha-WOLF 23 allele when heterozygously present in a spinach plant confers complete resistance to at least Peronospora effusa races Pe:3, Pfs:5, Pe:9, Pe:11 and Pe:14, intermediate resistance for Pe:8 and Pe:16 and does not confer resistance to at least Peronospora effusa races Pe:2, Pe:4, Pe:6, Pe:7, Pe:10, Pe:13, Pe:15 and Pe:18.

The invention further relates to a protein encoded by the allele of the invention. This protein is also referred to herein as the “protein of the invention” and confers downy mildew resistance to a spinach plant, in particular to at least Peronospora effusa races Pe:3, Pfs:5, Pe:8, Pe:9, Pe:11, Pe:14 and Pe:16, when the alpha-WOLF 23 encoding the protein of the invention is homozygously present in the genome of the spinach plant.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment. Suitably a comparison is made between nucleotide sequences that represent a gene that at least comprises a start codon and a stop codon or encodes an amino acid sequence which comprises a complete protein encoded by such a gene.

Sequence similarity for an amino acid sequence is calculated using EMBOSS stretcher 6.6.0 (www.ebi.ac.uk/Tools/psa/emboss_stretcher), using the EBLOSUM62 matrix with settings Gap open penalty: 12 and Gap extend penalty: 2. In case of DNA, sequence similarity is calculated using the DNA full matrix with settings Gap open penalty:16 and Gap extend penalty: 4.

The invention further relates to a plant, preferably a plant of the species Spinacia oleracea L., wherein the plant comprises the allele of the invention in its genome. A plant comprising the allele of the invention in its genome is referred to herein as a ‘plant of the invention’. In the context of this invention, a plant of the species Spinacia oleracea L. is a spinach plant.

In a further embodiment, the plant of the invention is an agronomically elite plant, preferably an agronomically elite spinach plant.

In the context of this invention, an agronomically elite plant is a plant having a genotype that, as a result of human intervention, comprises an accumulation of distinguishable and desirable agronomic traits which allow a producer to harvest a product of commercial significance, preferably the agronomically elite plant of the invention is a plant of an inbred line or a hybrid.

As used herein, a plant of an inbred line is a plant of a population of plants that is the result of three or more rounds of selfing, or backcrossing; or which plant is a double haploid. An inbred line may e.g. be a parent line used for the production of a commercial hybrid.

As used herein, a hybrid plant is a plant which is the result of a cross between two different plants having different genotypes. More in particular, a hybrid plant is the result of a cross between plants of two different inbred lines. Such a hybrid plant may e.g. be a plant of an F₁ hybrid variety.

The resistance of a spinach plant against one or more races of Peronospora effusa can be determined using a seedling test. Herein, a seedling test is defined as a test wherein spinach seeds are planted in trays containing growth medium, fertilized twice a week after seedling emergence. Plants are inoculated at the first true leaf stage with a sporangial suspension having a concentration of approximately 2.5×10⁵/ml of one of the pathogenic races of Peronospora effusa or isolates to be tested. Thirty plants per race are tested. The inoculated plants are placed in a dew chamber at 18° C. with 100% relative humidity for a 24 h period, and then moved to a growth chamber at 18° C. with a 12 h photoperiod for 6 days. After 6 days, the plants are returned to the dew chamber for 24 h to induce sporulation, and subsequently scored for a disease reaction.

As used herein, a plant is completely resistant against a Peronospora effusa race when a plant shows no symptoms in the seedling test described herein.

As used herein, a plant is intermediately resistant against a Peronospora effusa race when a plant shows only symptoms of chlorosis, or sporulation occurring only on the tips of the cotyledons in the seedling test described herein.

As used herein, a plant is susceptible to an isolate of a Peronospora effusa race when a plant shows more than only symptoms of chlorosis, or when sporulation occurs on an area larger than only the tips of the cotyledons in the seedling test described herein.

A plant carrying the alpha-WOLF 23 allele in heterozygous form may further comprise a beta-WOLF 0 allele on the homologous chromosome (as e.g. present in variety Viroflay) wherein the beta-WOLF 0 allele does not confer any resistance to downy mildew. However, a plant heterozygous for the alpha-WOLF 23 allele may further comprise an allele of the alpha or beta-WOLF gene on the homologous chromosome that does provide resistance to downy mildew. Preferably, such an allele would complement the alpha-WOLF 23 allele such that the spinach plant will be at least intermediately resistant to one or more other races to which the alpha-WOLF 23 allele does not provide resistance. Most preferably the other allele of the alpha or beta-WOLF gene complements the alpha-WOLF 23 allele such that the plant is resistant to Peronospora effusa races Pe:1 to Pe:19. In one embodiment such a plant is an agronomically elite plant.

Alternatively, the resistance profile of a plant carrying the alpha-WOLF 23 allele is complemented by a resistance conferring allele of a totally different gene. Examples of such genes are e.g. DMR1 as described in U.S. Pat. No. 8,354,570, DMR6 as described in U.S. Pat. No. 9,121,029 and p10 as described in U.S. Pat. No. 10,226,016.

The invention thus relates to a spinach plant carrying the alpha-WOLF 23 allele, and further comprising another genetic determinant, together resulting in resistance against Peronospora effusa races Pe:1 to Pe:19. The genetic determinant can be another resistance conferring alpha/beta-WOLF allele or a resistance conferring allele of a totally different gene or both.

Another aspect of the invention relates to a seed capable of growing into a plant of the invention wherein said plant comprises the allele of the invention. The invention also relates to use of said seed for the production of a plant of the invention, by growing said seed into a plant.

Yet another aspect of the invention relates to a leaf harvested from a spinach plant of the invention either in natural or processed form.

Spinach leaves are sold in packaged form, including without limitation as pre-packaged spinach leaves or as processed in a salad comprising said leaves. Mention of such a package is e.g. made in U.S. Pat. No. 5,523,136, which provides packaging film, and packages from such packaging film, including such packaging containing leafy produce, and methods for making and using such packaging film and packages, which are suitable for use with the spinach leaves of the invention. Thus, the invention comprises the use of and methods for making and using the leaves of the spinach plant of the invention, as well as leaves of spinach plants derived from the invention.

The invention further relates to a container which comprises one or more plants of the invention, or one or more spinach plants derived from a plant of the invention, in a growth substrate for harvest of leaves from the plant, in a domestic environment. This way the consumer may pick very fresh leaves for use in salads, when the plant is in a ready-to-harvest condition.

The invention also relates to propagation material suitable for producing a plant of the invention, wherein the propagation material is suitable for sexual reproduction, and is in particular selected from a microspore, a pollen, an ovary, an ovule, an embryo sac and an egg cell, or is suitable for vegetative reproduction, and is in particular selected from a cutting, a root, a stem a cell, and a protoplast, or is suitable for tissue culture of regenerable cells or protoplasts, and is in particular selected from a leaf, a pollen, an embryo, a cotyledon, a hypocotyl, a meristematic cell, a root, a root tip, an anther, a flower, a seed and a stem, wherein the propagation material comprises the allele of the invention.

The invention further relates to a cell of a plant of the invention. Such a cell may either be in isolated form, or a part of the complete plant or parts thereof and still forms a cell of the invention because such a cell comprises the allele of the invention. A cell of the invention may also be a regenerable cell that can regenerate into a new plant of the invention.

The invention further relates to plant tissue of a plant of the invention, which comprises the allele of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention further relates to a method for the production of a plant comprising the allele of the invention, which plant is resistant to Peronospora effusa, by using tissue culture or by using vegetative propagation.

Progeny of a plant, a cell, a tissue, or a seed of the invention, which progeny comprises the alpha-WOLF 23 allele also part of the invention. Such progeny can in itself be a plant, a cell, a tissue, or a seed. The progeny can in particular be progeny of a plant of the invention. As used herein, progeny comprises the first and all further descendants from a cross with a plant of the invention, wherein a cross comprises a cross with itself or a cross with another plant, and wherein a descendant that is determined to be progeny comprises the allele of the invention. Descendants can be obtained through selfing and/or further crossing. Progeny also encompasses material that is obtained by vegetative propagation or another form of multiplication.

The invention further relates to the germplasm of plants of the invention. The germplasm is constituted by all inherited characteristics of an organism and according to the invention encompasses at least the resistance trait of the invention. The germplasm can be used in a breeding program for the development of plants that show resistance to Peronospora effusa. The use of germplasm that comprises the allele of the invention in breeding is also part of the present invention. The invention further relates to seed capable of growing into a plant comprising the allele of the invention and being representative for the germplasm.

The invention also relates to the use of the alpha-WOLF 23 allele for producing a spinach plant that is resistant to Peronospora effusa.

The current invention also relates to the use of a plant of the invention as a crop, as a source of seed or as a source of propagation material.

The invention also relates to the use of a plant of the invention in breeding to confer resistance to Peronospora effusa.

The invention further relates to a method for seed production comprising growing a spinach plant from a seed of the invention that comprises the allele of the invention homozygously, allowing the plant to produce seed and harvesting the seed. Production of the seed is suitably done by selfing or by crossing with another plant that is optionally also a plant of the invention. The plant grown from the seed produced as described herein is resistant to Peronospora effusa races Pe:3, Pfs:5, Pe:8, Pe:9, Pe:11, Pe:14 and Pe:16.

The invention also relates to a method for producing a hybrid spinach seed, comprising crossing a first parent plant with a second parent plant and harvesting the resultant hybrid spinach seed, wherein the first parent plant and/or the second parent plant is a plant of the invention. Preferably, at least one of the parent plants comprises the allele of the invention homozygously.

In a particular embodiment, the first and/or second parent plant is a plant of an inbred line as defined herein.

The invention also relates to the hybrid seed produced by the method described herein and a hybrid plant grown from said hybrid seed, wherein said hybrid seed and plant comprise the allele of the invention.

Transgenic techniques used for transferring nucleotide sequences between plants that are sexually incompatible can also be used to produce a plant of the invention, by transferring the allele of the invention from one species to another. Techniques that can suitably be used comprise general plant transformation techniques known to the skilled person, such as the use of an Agrobacterium-mediated transformation method. A plant or a descendant thereof is a suitable source of the modified gene.

The invention further relates to a method for identifying a spinach plant comprising the allele of the invention, wherein the method comprises the following steps:
a) detecting the allele of the invention in the genome of a plant by determining the sequence of the allele, or
b) detecting the allele of the invention by determining the sequence of the LRR domain of the allele of the invention in the genome of a plant, or
c) by detecting a unique polymorphism in the allele of the invention.

Optionally, the method may further comprise testing of the plant comprising the allele of the invention for exhibiting resistance to Peronospora effusa.

The LRR domain of the allele of the invention can be determined by using a primer pair to amplify the LRR domain, wherein the forward primer is a nucleic acid molecule having the sequence of SEQ ID NO: 5 and wherein the reverse primer is a nucleic acid molecule having the sequence of SEQ ID NO: 6.

The invention further relates to a method for selecting a spinach plant resistant to Peronospora effusa, comprising identifying the presence of the allele of the invention, optionally testing the plant for resistance against Peronospora effusa, and selecting a plant comprising said allele as a plant which is resistant to at least Peronospora effusa races Pe:3, Pfs:5, Pe:9, Pe:11, and Pe:14, preferably to Pe:3, Pfs:5, Pe:8, Pe:9, Pe:11, Pe:14 and Pe:16.

Introduction of the allele of the invention can also be done through introgression from a plant comprising said allele, for example from a plant, or from progeny thereof, or from any other plant of the invention. Breeding methods such as crossing and selection, backcrossing, recombinant selection, or other breeding methods that result in the transfer of a genetic sequence from a resistant plant to a susceptible plant can be used. A resistant plant can be of the same species or of a different and/or wild species. Difficulties in crossing between species can be overcome through techniques known in the art such as embryo rescue, or cis-genesis can be applied instead. Progeny can be sexual or vegetative descendants, which can be selfed and/or crossed, and can be of an F1, F2, or further generation as long as the descendants still comprise the modified allele of the invention as present in seed. A plant produced by such method is also a part of the invention.

The invention also relates to a method for the production of a plant resistant to Peronospora effusa races Pe:3, Pfs:5, Pe:8, Pe:9, Pe:11, Pe:14 and Pe:16, comprising the steps of:
a) crossing a first parent plant comprising the allele of the invention with a second parent plant to obtain an F1 population;
b) optionally performing one or more rounds of selfing and/or crossing with a plant from the F1 population to obtain a further generation population;
c) selecting from the F1 population or further generation population a plant that comprises the allele of the invention as a resistant plant.

The invention also relates to a method for producing a plant which is resistant to Peronospora effusa races Pe:3, Pfs:5, Pe:8, Pe:9, Pe:11, Pe:14 and Pe:16, said method comprising:
a) crossing a first parent plant homozygously comprising the allele of the invention with a second parent plant;
b) backcrossing the plant resulting from step a) with the second parent plant for at least three generations;
c) selecting from the third or higher backcross population a plant that comprises at least the allele of the invention of the first parent plant of step a) as the plant which is resistant to Peronospora effusa.

The invention additionally provides for a method of introducing another desired trait into a plant that is resistant to Peronospora effusa, comprising:
a) crossing a plant comprising the allele of the invention with a second plant that comprises the other desired trait to produce F1 progeny;
b) selecting in the F1 for a plant that comprises the resistance and the other desired trait;
c) crossing the selected F1 progeny with one of the parents for at least three generations, to produce backcross progeny;
d) selecting backcross progeny comprising the resistance and the other desired trait; and
e) optionally repeating steps c) and d) one or more times in succession to produce selected fourth or higher backcross progeny that comprises the resistance and the other desired trait.

Optionally, selfing steps are performed after any of the crossing or backcrossing steps in above-described methods. Selection of a plant comprising the Peronospora effusa resistance and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method.

The present invention will be further illustrated in the Examples that follow and that are for illustration purposes only. The Examples are not intended to limit the invention in any way. In the Examples and in the application, reference is made to the following figures.

### Resistance Information

Resistance profile conferred by the alpha-WOLF 23 allele when heterozygously or homozygously present in a spinach plant. A “−” means complete resistance against a particular downy mildew race; “(−)” means intermediate resistance against a particular downy mildew race; “+” means that the allele confers no resistance and would cause a plant only carrying the alpha-WOLF 23 allele to be fully susceptible for that particular downy mildew race; “−*” means that when the allele is present homozygously it confers complete resistance against a particular downy mildew race, while the allele does confer intermediate resistance to that particular downy mildew race when present heterozygously; “nt” means that it has not been tested against that isolate.

TABLE 1
alpha-WOLF 23 resistance profile

| Peronospora effusa | Resistance |
|---|---|
| race | score |
| Pe:1 | nt |
| Pe:2 | + |
| Pe:3 | − |
| Pe:4 | + |
| Pe:5 | − |
| Pe:6 | + |
| Pe:7 | + |
| Pe:8 | −* |
| Pe:9 | − |
| Pe:10 | + |
| Pe:11 | − |
| Pe:12 | nt |
| Pe:13 | + |
| Pe:14 | − |
| Pe:15 | + |
| Pe:16 | −* |
| Pe:17 | nt |
| Pe:18 | + |
| Pe:19 | nt |

### Sequence Information

TABLE 2
Sequence information.

| | |
|---|---|
| SEQ ID NO: 1: | MAEIGYSVC |
| SEQ ID NO: 2: | KWMCLR |
| SEQ ID NO: 3: | HVGCVVDR |
| SEQ ID NO: 4: | DQEDEGEDN |
| SEQ ID NO: 5: | ACAAGTGGATGTGTCTTAGG |
| Forward primer | |
| LRR domain | |
| (Alpha) | |
| SEQ ID NO: 6: | TTCGCCCTCATCTTCCTGG |
| Reverse primer | |
| LRR domain | |
| (Alpha and | |
| Beta) | |
| SEQ ID NO: 7: | TCACGTGGGTTGTGTTGT |
| Forward primer | |
| LRR domain | |
| (Beta) | |
| SEQ ID NO: 8: | TCACGTGGGTTGTGTTGTCGATAGAGATCCAGAAATAGTCTT |
| Amplicon of | TTTATGTAGCAATAAGATTCGTTCGTATATTAGCGGTCGCTG |
| LRR domain of | CATAAAGAATCCGGTGGATTCACAAATAGACAACTGGATGT |
| the beta-WOLF | GCCTTAGGGTGTTGGACTTGTCAGATTCATGTGTTAAAGATT |
| 0 allele | TGTCTGATTCAATAGGTAAGCTGCTGCACTTAAGGTATCTTA |
| (Viroflay) | ACCTCTCTTCTAATATAAAGTTGGAGATAATCCCTGATGCAA |
| | TTACAAGACTGCATAACTTGCAGACACTACTTTTAGAAGATT |
| | GCAGAAGTTTAAAGGAGTTGCCAAAAGATTTTTGCAAATTG |
| | GTCAAACTGAGGCACTTGGAATTACAGGGTTGTCATGATTTG |
| | ATTGGTATGTCATTTGGAATGGATAAGCTAACTAGTCTTAGA |
| | ATACTACCAAACATTGTGGTGGGTAGGAAGGAACAAAGTGT |
| | TGATGATGAGCTGAAAGCCCTAAAAGGCCTCACCGAGATAA |
| | AAGGCTCCATTGATATCACAATCTATTCAAAATATAGAAGA |
| | GTTGAAGGCATGAATGGCACAGGAGGAGGAGCTGGGTATTT |
| | GAAGAGCATGAAACATCTCACGGGGGTTAATATTACATTTG |
| | ATGAAGGTGGATGTGTTAACCCTGAAGCTGTGTATTTGAAG |
| | AGCATGAAACATCTCACGAGGGTTATTATTATATTTGATTAT |
| | AAAGGTGGATGTGTTAACCCTGAAGCTGTGTTGGCAACCCT |
| | AGAGCCACCTTCAAATATCAAGAGGTTAGAGATGTGGCATT |
| | ACAGTGGTACAACAATTCCAGTATGGGGAAGAGCAGAGATT |
| | AATTGGGCAATCTCCCTCTCACATCTTGTCGACATCACGCTT |
| | GAAGATTGTTACAATTTGCAGGAGATGCCAGTGCTGAGTAA |
| | ACTGCCTCATTTGAAATCACTGGAACTTACAGAGTTGGATAA |
| | CTTAGAGTACATGGAGAGTAGAAGCAGCAGCAGTAGCAGTG |
| | ACACAGAAGCAGCAACACCAGAATTACCAACATTCTTCCCT |
| | TCCCTTGAAAAACTTACACTTTGGCGTCTGGACAAGTTGAAG |
| | GGTTTTGGGAACAGGAGATCGAGTAGTTTTCCCCGCCTCTCT |
| | AAATTGGAAATCTGGAAATGTCCAGATCTAACGTCATTTCCT |
| | TCTTGTCCAAGCCTTGAAGAGTTGGAATTGAAAGAAAACAA |
| | TGAAGCGTTGCAAATAATAGTAAAAATAACAACAACAAGAG |
| | GTAAAGAAGAAAAAGAAGAAGACAAGAATGCTGGTGTTGG |
| | AAATTCACAAGATGATGACAATGTCAAATTATGGAAGGTGG |
| | AAATAGACAATCTGGGTTATCTCAAATCACTGCCCACAAATT |
| | GTCTGACTCACCTCGACCTTACAATAAGTGATTCCAAGGAGG |
| | GGGAGGGTGAATGGGAAGTTGGGGATGCATTTCAGAAGTGT |
| | GTATCTTCTTTGAGAAGCCTCACCATAATCGGAAATCACGGA |
| | ATAAATAAAGTGAAGAGACTGTCTGGAAGAACAGGGTTGGA |
| | GCATTTCACTCTGTTGGAATCACTCAAACTTTCAGATATAGA |
| | AGACCAGGAAGATGAGGGCGAA |
| SEQ ID NO: 9: | HVGCVVDRDPEIVFLCSNKIRSYISGRCIKNPVDSQIDNWMCLR |
| Amino acid | VLDLSDSCVKDLSDSIGKLLHLRYLNLSSNIKLEIIPDAITRLHNL |
| sequence | QTLLLEDCRSLKELPKDFCKLVKLRHLELQGCHDLIGMSFGMD |
| encoded by | KLTSLRILPNIVVGRKEQSVDDELKALKGLTEIKGSIDITIYSKYR |
| amplicon of | RVEGMNGTGGGAGYLKSMKHLTGVNITFDEGGCVNPEAVYL |
| LRR domain | KSMKHLTRVIIIFDYKGGCVNPEAVLATLEPPSNIKRLEMWHYS |
| Beta Wolf 0 | GTTIPVWGRAEINWAISLSHLVDITLEDCYNLQEMPVLSKLPHL |
| (Viroflay) | KSLELTELDNLEYMESRSSSSSSDTEAATPELPTFFPSLEKLTLW |
| | RLDKLKGFGNRRSSSFPRLSKLEIWKCPDLTSFPSCPSLEELELK |
| | ENNEALQIIVKITTTRGKEEKEEDKNAGVGNSQDDDNVKLWK |
| | VEIDNLGYLKSLPTNCLTHLDLTISDSKEGEGEWEVGDAFQKC |
| | VSSLRSLTIIGNHGINKVKRLSGRTGLEHFTLLESLKLSDIEDQE |
| | DEGE |
| SEQ ID NO: | ATGGCCGAAATCGGATACTCGGTTTGTGCGAAACTCATCGA |
| 10: Coding | AGTGATTGGCAGTGAGCTGATCAAAGAGATTTGCGACACAT |
| sequence of the | GGGGTTACAAATCTCTTCTTGAGGACCTCAACAAAACTGTAT |
| alpha-WOLF 23 | TGACGGTCAGGAACGTTCTCATTCAGGCCGGGGTGATGCGG |
| allele | GAGCTTACTAGTGAACAACAAGGTTTCATTGCAGACCTTAA |
| | AGATGTTGTTTATGATGCTGATGACTTGTTCGACAAGTTACT |
| | CACTCGTGCTGAGCGAAAACAGATTGATGGAAACGAAATCT |
| | CTGAAAAGGTACGTCGTTTCTTTTCCTCTAGTAACAAGATCG |
| | GTCAAGCTTACTACATGTCTCGTAAGGTTAAGGAAATTAAG |
| | AAGCAGTTGGATGAAATTGTTGATAGGCATACAAAATTTGG |
| | GTTTAGTGCTGAGTTTATACCTGTTTGTAGGGAAAGGGGGAA |
| | CGAGAGGGAAACACGTTCATATATAGATGTCAAGAATATTC |
| | TTGGGAGGGATAAAGATAAGAATGATATCATAGATAGGTTG |
| | CTTAATCGTAATGATAATGAAGCTTGTAGTTTCCTGACCATA |
| | GTGGGAGCGGGAGGATTGGGAAAAACTGCTCTTGCCCAACT |
| | TGTGTTCAATGATGAAAGGGTCAAAATTGAGTTTCATGATTT |
| | GAGGTATTGGGTTTGTGTCTCTGATCAAGATGGGGGCCAATT |
| | TGATGTGAAAGAAATCCTTTGTAAGATTTTAGAGGTGGTTAC |
| | TAAGGAGAAAGTTGATAATAGTTCCGCATTGGAATTGGTAC |
| | AAAGCCAATTTCAAGAGAAGTTAAGAGGAAAGAAGTACTTC |
| | CTTGTTCTTGATGATGTATGGAACGAGGATCGTGAGAAGTG |
| | GTTTAAATTGGAAGAGTTGTTAATGTTGGGTCAAGGGGGAA |
| | GCAAGGTTGTAGTGACCGCACGTTCAGAGAAGACAGCAAAT |
| | GTCATAGGGAAAAGACATTTTTATACACTGGAATGTTTGTCG |
| | CCAGATTATTCATGGAGCTTATTTGAAATGTCGGCTTTTCAG |
| | AAAGGGCATGAGCAGGAAAACCATGACGAACTAGTTGATAT |
| | TGGGAAAAAGATTGTTGAAAAATGTTATAACAATCCACTTG |
| | CTATAACGGTGGTAGGAAGTCTTCTTTATGGAGAGGAGATA |
| | AGTAAGTGGCGGTCATTTGAAATGAGTGAGTTGGCCAAAAT |
| | TGGCAATGGGGATAACAAGATTTTGTCGATATTGAAGCTCA |
| | GTTACTACAATCTTGCAAACTCTTTGAAGAGTTGTTTTAGTT |
| | ATTGTGCAGTGTTTCCCAAGGATCATGAAATAAAGAAGGAG |
| | ATGTTGATTGAACTTTGGATGGCACAAGGATATGTTGTGCCG |
| | TTGGATGGAGGTCAAAGTATAGAAGATGCTGCCGAGGAACA |
| | TTTTGTAATTTTGTTACGAAGGTGTTTCTTTCAAGATGTAGTG |
| | AAGGATGAATACGGTGATGTTGATTCTGTTAAAATCCACGA |
| | CTTGATGCACGATGTCGCCCAAGAAGTGGGCAGAGAGGAAA |
| | TCTGTATAGTGAATGCTAATACAAAGAACTTGGGTGATAAA |
| | ATCCGTCATGTACATTGTGATGTCAATAGATATGCACAAAGA |
| | GTCTCTCTGTGTAGCCATAAGATTCGTTCGTATATTGGTGGT |
| | CAATGTGAAAAACGTTGGGTGGATACACTAATAGACAAGTG |
| | GATGTGTCTTAGGGTGTTGGACTTGTCAAGGTCGGATGTTAA |
| | AAATTTGCCTAATTCAATAGGTAAATTGTTGCACTTGAGGTG |
| | TCTTAACCTGTCTTATAATGATCTGTTGATACTCCCTGATGCA |
| | ATTACAAGACTGCATAATTTGCAGACACTGCTTTTAAAAGAT |
| | TGCGGAAGTTTAATGGAGTTGCCAAAAGATTTTTGCAAATTG |
| | GTCAAACTGAGGCACTTGGAATTACAGGGTTGTCATGATTTG |
| | ATTGGTATGCCATTGGGAATGGATAGGCTAACTAGTCTTAGA |
| | GTACTGCCATTCTTTGTGGTGGGTAGGAAGGAACAAAGTGTT |
| | GATGATGAGCTGAAAGCCCTAAAAGGCCTCACCGAGATAAA |
| | AGGCTCCATTCGTATTAGAATCCATTCAAAGTATAGAATAGT |
| | TGAAGGCATGAATGACACAGGAGGAGCTGGGTATTTGAAGA |
| | GCATGAAACATCTCACGGGGGTTAATATTAGATTTGATGATA |
| | GAGAAGGTGTATTTGTTAACCCTGAAGCTGTGTTGGCAACCC |
| | TAGAGCCACCTTCAAATATCAAGAGGTTAGAGGTGTGGCAT |
| | TACGATGGTACAACAATTCCAGTATGGGGAAGAGCAGAGAT |
| | TAATTGGGCAATCTCCCTCTCGCATCTTGTTGACATCGAGCT |
| | TTGGCGTTGTAGTAATTTGCAGGAGATGCCAGTGCTGAGTAA |
| | ACTGCCTCATTTGAAATCACTGAAACTTGAAGATTTGAATAA |
| | CTTAGAGTACATGGAGAGTAGAAGCAGCAGCAGTAGCAGTG |
| | ACACAGAAGCAGCAACACCAGAATTACCAACATTCTTCCCT |
| | TCCCTTGAAAAACTTACACTTTGGCGTCTGAACAAGTTGAAG |
| | AGTTTTGGGAACAGGAGATCGAGTAGTTTTCCCCGCCTCTCT |
| | GAATTGGAAATCTGGGAATGCCCAGATCTAACGTGGTTTCCT |
| | CCCTGTCCAAGCCTTGAAGAGTTGACATTGAAAGACAACCA |
| | TGAAGCGTTGCAAATAATAGTAAAAATAACAACAACAAGAG |
| | GTAAAGAAGAAAAAGAAGAAGACAAGAATGCTGGTGTTGG |
| | AAATTCACAAGATGATGACAATGTCAAATTACGGAAGGTGG |
| | AAATAGACAATCTGGGTTATCTCAAATCACTGCCCACAAATT |
| | GTCTTACTCACCTCGACCTTACAATAAGTGATTCCAAGGAGG |
| | GGGAGGGTGAATGGGAAGTTGGGGATGCATTTCAGAAGTGT |
| | GTATCTTCTTTGAGAAAGCTCAGCATAATCGGAAATCACGG |
| | AATAAATAAAGTGAAGAGACTGTCTGGAAGAACAGGGTTGG |
| | AGCATTTCACTCTGTTGGACTCACTCGAACTTTCAAATATAG |
| | AAGACCAGGAAGATGAGGGCGAAGACAACATCATATTCTGG |
| | AAATCCTTTCCTCAAAACCTCCGCAGTTTGGAAATTGAAGAC |
| | TCTGACAAAATGACAAGTTTGCCCATGGGGATGCAGTACTT |
| | AACCTCCCTCCAAACCCTCGAACTATCATATTGTGATGAATT |
| | GAATTCCCTTCCAGAATGGATAAGCAGCTTATCATCTCTTCA |
| | ATACCTGGGCATATTCAACTGTCCAGCCCTGAAATCACTACC |
| | AGAAGCAATGCGGAACCTCACCTCCCTTCAGACACTTGGGA |
| | TATCGGATTGTCCAGACCTAGTTAAAATATGCAGAAAACCC |
| | AACGGCGAGGACTATCCCAAAATTCAATACATCCCCAAAAT |
| | TATTATATGGTAG |
| SEQ ID NO: | MAEIGYSVCAKLIEVIGSELIKEICDTWGYKSLLEDLNKTVLTV |
| 11: Amino acid | RNVLIQAGVMRELTSEQQGFIADLKDVVYDADDLFDKLLTRAE |
| sequence of the | RKQIDGNEISEKVRRFFSSSNKIGQAYYMSRKVKEIKKQLDEIV |
| alpha-WOLF 23 | DRHTKFGFSAEFIPVCRERGNERETRSYIDVKNILGRDKDKNDII |
| allele | DRLLNRNDNEACSFLTIVGAGGLGKTALAQLVENDERVKIEFH |
| | DLRYWVCVSDQDGGQFDVKEILCKILEVVTKEKVDNSSALELV |
| | QSQFQEKLRGKKYFLVLDDVWNEDREKWFKLEELLMLGQGG |
| | SKVVVTARSEKTANVIGKRHFYTLECLSPDYSWSLFEMSAFQK |
| | GHEQENHDELVDIGKKIVEKCYNNPLAITVVGSLLYGEEISKWR |
| | SFEMSELAKIGNGDNKILSILKLSYYNLANSLKSCFSYCAVFPK |
| | DHEIKKEMLIELWMAQGYVVPLDGGQSIEDAAEEHFVILLRRC |
| | FFQDVVKDEYGDVDSVKIHDLMHDVAQEVGREEICIVNANTK |
| | NLGDKIRHVHCDVNRYAQRVSLCSHKIRSYIGGQCEKRWVDTL |
| | IDKWMCLRVLDLSRSDVKNLPNSIGKLLHLRCLNLSYNDLLILP |
| | DAITRLHNLQTLLLKDCGSLMELPKDFCKLVKLRHLELQGCHD |
| | LIGMPLGMDRLTSLRVLPFFVVGRKEQSVDDELKALKGLTEIK |
| | GSIRIRIHSKYRIVEGMNDTGGAGYLKSMKHLTGVNIRFDDREG |
| | VFVNPEAVLATLEPPSNIKRLEVWHYDGTTIPVWGRAEINWAIS |
| | LSHLVDIELWRCSNLQEMPVLSKLPHLKSLKLEDLNNLEYMES |
| | RSSSSSSDTEAATPELPTFFPSLEKLTLWRLNKLKSFGNRRSSSFP |
| | RLSELEIWECPDLTWFPPCPSLEELTLKDNHEALQIIVKITTTRG |
| | KEEKEEDKNAGVGNSQDDDNVKLRKVEIDNLGYLKSLPTNCL |
| | THLDLTISDSKEGEGEWEVGDAFQKCVSSLRKLSIIGNHGINKV |
| | KRLSGRTGLEHFTLLDSLELSNIEDQEDEGEDNIIFWKSFPQNLR |
| | SLEIEDSDKMTSLPMGMQYLTSLQTLELSYCDELNSLPEWISSL |
| | SSLQYLGIFNCPALKSLPEAMRNLTSLQTLGISDCPDLVKICRKP |
| | NGEDYPKIQYIPKIIIW |
| SEQ ID NO: | TGGATGTGTCTTAGGGTGTTGGACTTGTCAAGGTCGGATGTT |
| 12: Amplicon | AAAAATTTGCCTAATTCAATAGGTAAATTGTTGCACTTGAGG |
| of LRR domain | TGTCTTAACCTGTCTTATAATGATCTGTTGATACTCCCTGATG |
| of the alpha- | CAATTACAAGACTGCATAATTTGCAGACACTGCTTTTAAAAG |
| WOLF 23 allele | ATTGCGGAAGTTTAATGGAGTTGCCAAAAGATTTTTGCAAAT |
| | TGGTCAAACTGAGGCACTTGGAATTACAGGGTTGTCATGATT |
| | TGATTGGTATGCCATTGGGAATGGATAGGCTAACTAGTCTTA |
| | GAGTACTGCCATTCTTTGTGGTGGGTAGGAAGGAACAAAGT |
| | GTTGATGATGAGCTGAAAGCCCTAAAAGGCCTCACCGAGAT |
| | AAAAGGCTCCATTCGTATTAGAATCCATTCAAAGTATAGAAT |
| | AGTTGAAGGCATGAATGACACAGGAGGAGCTGGGTATTTGA |
| | AGAGCATGAAACATCTCACGGGGGTTAATATTAGATTTGAT |
| | GATAGAGAAGGTGTATTTGTTAACCCTGAAGCTGTGTTGGCA |
| | ACCCTAGAGCCACCTTCAAATATCAAGAGGTTAGAGGTGTG |
| | GCATTACGATGGTACAACAATTCCAGTATGGGGAAGAGCAG |
| | AGATTAATTGGGCAATCTCCCTCTCGCATCTTGTTGACATCG |
| | AGCTTTGGCGTTGTAGTAATTTGCAGGAGATGCCAGTGCTGA |
| | GTAAACTGCCTCATTTGAAATCACTGAAACTTGAAGATTTGA |
| | ATAACTTAGAGTACATGGAGAGTAGAAGCAGCAGCAGTAGC |
| | AGTGACACAGAAGCAGCAACACCAGAATTACCAACATTCTT |
| | CCCTTCCCTTGAAAAACTTACACTTTGGCGTCTGAACAAGTT |
| | GAAGAGTTTTGGGAACAGGAGATCGAGTAGTTTTCCCCGCC |
| | TCTCTGAATTGGAAATCTGGGAATGCCCAGATCTAACGTGGT |
| | TTCCTCCCTGTCCAAGCCTTGAAGAGTTGACATTGAAAGACA |
| | ACCATGAAGCGTTGCAAATAATAGTAAAAATAACAACAACA |
| | AGAGGTAAAGAAGAAAAAGAAGAAGACAAGAATGCTGGTG |
| | TTGGAAATTCACAAGATGATGACAATGTCAAATTACGGAAG |
| | GTGGAAATAGACAATCTGGGTTATCTCAAATCACTGCCCAC |
| | AAATTGTCTTACTCACCTCGACCTTACAATAAGTGATTCCAA |
| | GGAGGGGGAGGGTGAATGGGAAGTTGGGGATGCATTTCAGA |
| | AGTGTGTATCTTCTTTGAGAAAGCTCAGCATAATCGGAAATC |
| | ACGGAATAAATAAAGTGAAGAGACTGTCTGGAAGAACAGG |
| | GTTGGAGCATTTCACTCTGTTGGACTCACTCGAACTTTCAAA |
| | TATAGAAGACCAGGAAGATGAGGGCGAA |
| SEQ ID NO: | WMCLRVLDLSRSDVKNLPNSIGKLLHLRCLNLSYNDLLILPDAI |
| 13: Amino acid | TRLHNLQTLLLKDCGSLMELPKDFCKLVKLRHLELQGCHDLIG |
| sequence | MPLGMDRLTSLRVLPFFVVGRKEQSVDDELKALKGLTEIKGSIR |
| encoded by | IRIHSKYRIVEGMNDTGGAGYLKSMKHLTGVNIRFDDREGVFV |
| amplicon of the | NPEAVLATLEPPSNIKRLEVWHYDGTTIPVWGRAEINWAISLSH |
| alpha-WOLF 23 | LVDIELWRCSNLQEMPVLSKLPHLKSLKLEDLNNLEYMESRSSS |
| allele | SSSDTEAATPELPTFFPSLEKLTLWRLNKLKSFGNRRSSSFPRLS |
| | ELEIWECPDLTWFPPCPSLEELTLKDNHEALQIIVKITTTRGKEE |
| | KEEDKNAGVGNSQDDDNVKLRKVEIDNLGYLKSLPTNCLTHL |
| | DLTISDSKEGEGEWEVGDAFQKCVSSLRKLSIIGNHGINKVKRL |
| | SGRTGLEHFTLLDSLELSNIEDQEDEGE |

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1—Testing for Resistance to Peronospora effusa in Spinach Plants

The resistance to downy mildew infection was assayed as described by Irish et al. (2008; Phytopathol. 98: 894-900), using a differential set. Spinach plants of the invention were sown along with spinach plants from different other genotypes (see Table 3) in trays containing Scotts Redi-Earth medium, and fertilized twice a week after seedling emergence with Osmocote Peter's (13-13-13) fertilizer (Scotts). Plants were inoculated with a sporangial suspension (2.5×10⁵/ml) of a pathogenic race of Peronospora effusa at the first true leaf stage. In this manner, 4 officially recognized pathogenic race were tested.

The inoculated plants were placed in a dew chamber at 18° C. with 100% relative humidity for a 24 h period, and then moved to a growth chamber at 18° C. with a 12 h photoperiod for 6 days. After 6 days, the plants were returned to the dew chamber for 24 h to induce sporulation, and they were scored for disease reaction.

Plants for this specific test were scored as resistant, intermediately resistant, or susceptible based on symptoms of chlorosis and signs of pathogen sporulation on the cotyledons and true leaves, as described by Irish et al. (2007; Plant Dis. 91: 1392-1396). Plants exhibiting no evidence of chlorosis and sporulation were in this specific test considered as resistant. Resistant plants were re-inoculated to assess whether plants initially scored as resistant had escaped infection, or whether they were truly resistant. Plants that showed only symptoms of chlorosis, or sporulation occurring only on the tips of the cotyledons were scored as intermediately resistant. Plants showing more than these symptoms of downy mildew infection were scored as being susceptible.

Table 1 shows the resistance of a plant carrying the alpha-WOLF 23 allele to each one of these pathogenic races. Table 3 shows the differential set of spinach downy mildew races and the resistance of various spinach varieties (hybrids) to each one of these pathogenic races. A susceptible reaction is scored as “+” (indicating a successful infection by the fungus, with sporulation occurring on the entire cotyledon), and resistance is depicted as “−” (absence of sporulation on the cotyledons). A weak resistance response is indicated as “(−)”, which in practice means a slightly reduced level of infection (with only symptoms of chlorosis, or sporulation only occurring on the tips of the cotyledons in the differential seedling test).

TABLE 3

| Races/plants | Viroflay | NIL 5 | NIL 3 | NIL 4 | NIL 6 | NIL 1 | NIL 2 | Pigeon | Caladonia | Meerkat | Hydrus |
|---|---|---|---|---|---|---|---|---|---|---|---|
| Pe: 1 | + | − | − | − | − | − | − | − | | − | − |
| Pe: 2 | + | − | + | − | + | − | − | − | | − | − |
| Pe: 3 | + | + | − | − | − | − | − | − | | − | − |
| Pe: 4 | + | + | + | − | − | − | − | − | | − | − |
| Pe: 5 | + | + | − | + | − | − | − | − | | − | − |
| Pe: 6 | + | + | + | + | + | − | − | − | | − | − |
| Pe: 7 | + | + | + | + | − | − | − | − | | − | − |
| Pe: 8 | + | + | − | + | + | + | − | − | | − | − |
| Pe: 9 | + | + | − | + | + | − | − | − | | − | − |
| Pe: 10 | + | + | + | + | + | + | − | − | | − | − |
| Pe: 11 | + | + | − | + | − | − | + | − | | − | − |
| Pe: 12 | + | + | − | + | + | + | + | − | | − | − |
| Pe: 13 | + | + | + | + | − | (−) | + | − | | − | − |
| Pe: 14 | + | + | − | + | + | + | + | + | | − | − |
| Pe: 15 | + | + | + | − | − | − | − | − | + | − | − |
| Pe: 16 | + | + | − | + | − | − | + | + | − | + | − |
| Pe: 17 | + | + | + | + | + | + | + | + | + | (−) | − |
| Pe: 18 | + | + | + | + | | | + | + | + | + | − |
| Pe: 19 | + | + | − | + | + | + | + | + | − | + | + |

### Example 2—Amplification of the LRR Domain-Encoding Region

The isolated genomic DNA of a spinach plant comprising the alpha-WOLF 23 allele was used in polymerase chain reactions (PCR), using forward primer ACAAGTGGATGTGTCTTAGG (SEQ ID NO: 5) and reverse primer TTCGCCCTCATCTTCCTGG (SEQ ID NO: 6). The primer pair amplifies the LRR domain-encoding region of an alpha-WOLF gene, and has been designed for selectively amplifying part of a WOLF gene, and not of other CC-NBS-LRR protein-encoding genes.

PCR conditions for amplifying the LRR domain-encoding region of an alpha-WOLF gene using primers having SEQ ID NO: 5 and SEQ ID NO: 6 were as follows, using Platinum Taq enzyme (Thermo Fisher Scientific):
3 minutes at 95° C. (initial denaturing step)
40 amplification cycles, each cycle consisting of: 30 seconds denaturation at 95° C., 30 seconds annealing at 60° C., and 30 seconds extension at 72° C.
2 minutes at 72° C. (final extension step)

The isolated genomic DNA of a spinach plant of variety Viroflay comprising the beta-WOLF 0 allele was used in polymerase chain reactions (PCR), using forward primer TCACGTGGGTTGTGTTGT (SEQ ID NO: 7) and reverse primer TTCGCCCTCATCTTCCTGG (SEQ ID NO: 6). The primer pair amplifies the LRR domain-encoding region of a beta-WOLF gene, and has been designed for selectively amplifying part of a WOLF gene, and not of other CC-NBS-LRR protein-encoding genes.

PCR conditions for amplifying the LRR domain-encoding region of a beta-WOLF gene using primers having SEQ ID NO: 6 and SEQ ID NO: 7 were as follows, using Platinum Taq enzyme (Thermo Fisher Scientific):
3 minutes at 95° C. (initial denaturing step)
40 amplification cycles, each cycle consisting of: 30 seconds denaturation at 95° C., 50 seconds annealing at 58° C. and 50 seconds extension at 72° C.
2 minutes at 72° C. (final extension step)

The PCR products were visualized on agarose gel (not shown), and DNA was purified from the PCR reaction. Subsequently the sequence of the PCR products was determined using methods well known in the art.

The DNA sequence of the LRR domain of the alpha-WOLF 23 allele amplified by primers having SEQ ID NO: 5 and SEQ ID NO: 6 is provided in Table 2 under SEQ ID NO: 12.

The DNA sequence of the LRR domain of the beta-WOLF 0 allele amplified by primers having SEQ ID NO: 6 and SEQ ID NO: 7 is provided in Table 2 under SEQ ID NO: 8.

Finally, the obtained sequences were translated into the corresponding amino acid sequence of the LRR domain having SEQ ID NO: 13 and SEQ ID NO: 9 for the alpha-WOLF 23 allele and the beta-WOLF 0, respectively (See also Table 2).

If PCR products were to be sequenced using SMRT sequencing (Pacific Biosciences), PCR primers and PCR conditions were different.

To the above-mentioned forward primers, the following standard amplification sequence was added: GCAGTCGAACATGTAGCTGACTCAGGTCAC.

To the reverse primer, the following standard amplification sequence was added:

TGGATCACTTGTGCAAGCATCACATCGTAG.

### Example 3—Introducing an Alpha-WOLF 23 Allele in a Plant not Carrying the Allele

A spinach plant comprising the alpha-WOLF 23 allele was crossed with a plant of variety Viroflay carrying the beta-WOLF 0 allele to obtain a F1 generation. Subsequently, a F1 plant was selfed to obtain a F2 population.

Plants of the F2 population were assayed as described in Example 1 for resistance to Peronospora effusa Pe:3, Pe:5, Pe:9, Pe:11 and Pe:14. Approximately 75% of the plants scored completely resistant in the assay. This segregation pattern is consistent with that of a dominant inheritance.

Genomic DNA of each plant of the same F2 population was isolated and used in two different polymerase chain reactions (PCR). The first PCR reaction was done using primers for amplifying the LRR domain of an alpha-WOLF allele and the second PCR reaction was done using primers for amplifying the LRR domain of a beta-WOLF allele, both as described in Example 2.

The PCR products were visualized on agarose gel (not shown), this demonstrated that approximately 75% of the plants contained an alpha-WOLF fragment, and that the remaining approximately 25% of the plants only contained a beta-WOLF fragment. The plants containing the alpha-WOLF fragment completely correlated with the plants that scored resistant for Pe:3, Pe:5, Pe:9, Pe:11 and Pe:14. The plants only comprising the beta-WOLF fragment completely correlated with the plants that scored susceptible for Pe:3, Pe:5, Pe:9, Pe:11 and Pe:14.

DNA from the PCR reaction was purified, and subsequently the sequence of the PCR products was determined. The alpha-WOLF PCR products gave a sequence that corresponded to the sequence of SEQ ID NO: 12, the sequence of the LRR domain of the alpha-WOLF 23 allele. The beta-WOLF PCR products gave a sequence that corresponded to the sequence of SEQ ID NO: 8 the sequence of the LRR domain of the beta-WOLF 0 allele.

The invention is further described by the following numbered paragraphs:

1. An allele of an alpha-WOLF gene encoding a protein which confers at least resistance to Peronospora effusa races Pe:3, Pfs:5, Pe:8, Pe:9, Pe:11, Pe:14 and Pe:16 and does not confer resistance to races Pe:2, Pe:4, Pe:6, Pe:7, Pe:10, Pe:13, Pe:15 and Pe:18 when expressed in a spinach plant, and wherein the protein comprises in its amino acid sequence the motif “MAEIGYSVC” (SEQ ID NO: 1) at its N-terminus, and the motif “KWMCLR” (SEQ ID NO: 2); and wherein said allele comprises:
a) a nucleotide sequence comprising a coding sequence that has at least 97% sequence identity to SEQ ID NO: 10, or
b) a nucleotide sequence encoding a protein which has an amino acid sequence that has at least 93.5% sequence similarity to SEQ ID NO: 11, or
c) a nucleotide sequence encoding an LRR domain which nucleotide sequence has at least 94.5% sequence identity to SEQ ID NO: 12, or
d) a nucleotide sequence encoding an LRR domain which has an amino acid sequence that has at least 93% sequence similarity to SEQ ID NO: 13.

2. The allele of paragraph 1, wherein the protein encoded by the allele further comprises the motif “DQEDEGEDN” (SEQ ID NO: 4).

3. The allele or paragraphs 1 or 2, wherein the allele confers complete resistance to at least Pe:3, Pfs:5, Pe:9, Pe:11, and Pe:14 when homozygously present in a spinach plant.

4. A protein encoded by the allele of any one of paragraphs 1 to 3.

5. A spinach plant comprising the allele of any one of paragraphs 1 to 3.

6. The spinach plant of paragraph 5, wherein the plant is an agronomically elite plant, in particular a hybrid variety or an inbred line.

7. A spinach seed capable of growing into the spinach plant of paragraph 5 or 6.

8. Propagation material suitable for producing the spinach plant of paragraph 5 or 6, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the allele of any one of paragraphs 1 to 3.

9. Method for identifying a spinach plant comprising the allele of any one of paragraphs 1 to 3, wherein the method comprises the following steps:
a) detecting the allele of paragraph 1 or 2 in the genome of a plant by determining the sequence of the allele, or
b) detecting the sequence of the LRR domain of the allele of any one of paragraphs 1 to 3 in the genome of a plant, or
c) by detecting a unique polymorphism in the allele of any one of paragraphs 1 to 3, and optionally
d) testing of the plant comprising the allele of the invention for exhibiting resistance to Peronospora effusa.

10. The method of paragraph 9, wherein the LRR domain is determined by using a primer pair to amplify the LRR domain, wherein the forward primer is a nucleic acid molecule having the sequence of SEQ ID NO: 5.

11. The method of paragraph 9, wherein the LRR domain is determined by using a primer pair to amplify the LRR domain, wherein the reverse primer is a nucleic acid molecule having the sequence of SEQ ID NO: 6.

12. A method for selecting a spinach plant resistant to Peronospora effusa, comprising identifying the presence of the allele of any one of paragraphs 1 to 3, optionally testing the plant for resistance against Peronospora effusa, and selecting a plant comprising said allele as a plant which is resistant to at least Peronospora effusa races Pe:3, Pfs:5, Pe:9, Pe:11, and Pe:14, preferably to Pe:3, Pfs:5, Pe:8, Pe:9, Pe:11, Pe:14 and Pe:16.

13. A method for producing a spinach plant resistant to Peronospora effusa races Pe:3, Pfs:5, Pe:8, Pe:9, Pe:11, Pe:14 and Pe:16 comprising the steps of:
a) crossing a first parent plant comprising the allele of any one of paragraphs 1 to 3 with a second parent plant to obtain an F1 population;
b) optionally performing one or more rounds of selfing and/or crossing with a plant from the F1 population to obtain a further generation population;
c) selecting from the F1 population or the further generation population a plant that comprises the allele of any one of paragraphs 1 to 3 as a resistant plant.

14. A method for producing hybrid spinach seed resistant to Peronospora effusa, comprising the steps of crossing a first parent plant with a second parent plant, wherein one or both parent plants are homozygous for the allele of any one of paragraphs 1 to 3 and harvesting the hybrid seed.

15. The hybrid seed produced by the method of paragraph 14.

16. A plant grown from the hybrid seed of paragraph 15.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A spinach plant comprising an allele of an alpha-WOLF gene encoding a protein which confers resistance comprising at least resistance to Peronospora effusa races Pe:3, Pfs:5, Pe: 8, Pe:9, Pe:11, Pe:14 and Pe:16 and does not confer resistance to races Pe:2, Pe:4, Pe:6, Pe:7, Pe:10, Pe:13, Pe:15 and Pe:18, wherein the protein comprises in its amino acid sequence the motif MAEIGYSVC(SEQ ID NO: 1) at its N-terminus, and the motif KWMCLR (SEQ ID NO: 2); and wherein said allele comprises:
a) a nucleotide sequence comprising a coding sequence having at least 97% sequence identity to SEQ ID NO: 10; or
b) a nucleotide sequence encoding a protein having an amino acid sequence having at least 93.5% sequence identity to SEQ ID NO: 11; or
c) a nucleotide sequence encoding an LRR domain, wherein the nucleotide sequence has at least 94.5% sequence identity to SEQ ID NO: 12; or
d) a nucleotide sequence encoding an LRR domain, wherein the LRR domain comprises an amino acid sequence having at least 93% sequence similarity to SEQ ID NO: 13.

2. The spinach plant of claim 1, wherein the protein encoded by the allele further comprises the motif DQEDEGEDN (SEQ ID NO: 4).

3. The spinach plant of claim 1, wherein when homozygously present, the allele confers complete resistance to at least Pe:3, Pfs:5, Pe:9, Pe:11, and Pe:14.

4. The spinach plant of claim 1, wherein the allele of the alpha-WOLF gene comprises the nucleotide sequence comprising the coding sequence having at least 97% sequence identity to SEQ ID NO: 10.

5. The spinach plant of claim 1, wherein the allele of the alpha-WOLF gene comprises the nucleotide sequence encoding the protein which has the amino acid sequence having at least 93.5% sequence identity to SEQ ID NO: 11.

6. The spinach plant of claim 1, wherein the allele of the alpha-WOLF gene comprises the nucleotide sequence encoding the LRR domain, wherein the nucleotide sequence has at least 94.5% sequence identity to SEQ ID NO: 12.

7. The spinach plant of claim 1, wherein the allele of the alpha-WOLF gene comprises the nucleotide sequence encoding the LRR domain, wherein the LRR domain comprises the amino acid sequence having at least 93% sequence similarity to SEQ ID NO: 13.

8. Propagation material from or suitable for producing the plant of claim 1, wherein the propagation material is suitable for sexual reproduction, and comprises a microspore, pollen, ovary, ovule, embryo sac or an egg cell, or the propagation material is suitable for vegetative reproduction and comprises a cutting, root, stem cell, or a protoplast; or the propagation material is suitable for tissue culture of regenerable cells or protoplasts, and comprises a leaf, pollen, embryo, cotyledon, hypocotyl, meristematic cell, root, root tip, anther, flower or a stem; and wherein the propagation material comprises the allele of the alpha-WOLF gene.

9. A method for identifying a spinach plant comprising an allele of an alpha-WOLF gene encoding a protein which confers resistance comprising resistance to at least Peronospora effusa races Pe:3, Pfs:5, Pe:8, Pe:9, Pe:11, Pe:14 and Pe:16 and does not confer resistance to races Pe:2, Pe:4, Pe:6, Pe:7, Pe:10, Pe:13, Pe:15 and Pe:18, wherein:
the allele of the alpha-WOLF gene comprises:
i) a nucleotide sequence comprising a coding sequence having at least 97% sequence identity to SEQ ID NO: 10 (nucleotide sequence (i)), or
ii) a nucleotide sequence encoding a protein which has an amino acid sequence having at least 93.5% sequence identity to SEQ ID NO: 11 (nucleotide sequence (i)); or
iii) a nucleotide sequence encoding an LRR domain wherein the nucleotide sequence has at least 94.5% sequence identity to SEQ ID NO: 12 (nucleotide sequence (iii)); or
iv) a nucleotide sequence encoding an LRR domain, wherein the LRR domain comprises an amino acid sequence having at least 93% sequence similarity to SEQ ID NO: 13 (nucleotide sequence (iv)); and
the protein encoded by the allele of the alpha-WOLF gene comprises in its amino acid sequence the motif MAEIGYSVC (SEQ ID NO: 1) at its N-terminus, and the motif KWMCLR (SEQ ID NO: 2),
the method comprises:
detecting by genetic analysis in the genome of the spinach plant nucleotide (i), or nucleotide (ii), or nucleotide (iii), or nucleotide (iv), or a unique polymorphism in the allele of the alpha-WOLF gene, thereby identifying the spinach plant comprising the allele of the alpha-WOLF gene;
and optionally
testing the plant identified as comprising the allele of the alpha-WOLF gene for exhibiting resistance to Peronospora effuse.

10. The method of claim 9, wherein the method comprises determining the presence of the nucleotide sequence encoding the LRR domain by using a primer pair comprising a forward primer and a reverse primer to amplify the nucleotide sequence encoding the LRR domain, wherein the forward primer is a nucleic acid molecule having the sequence of SEQ ID NO: 5.

11. The method of claim 9, wherein the method comprises determining the presence of the nucleotide sequence encoding the LRR domain by using a primer pair comprising a forward primer and a reverse primer to amplify the nucleotide sequence encoding the LRR domain, wherein the reverse primer is a nucleic acid molecule having the sequence of SEQ ID NO: 6.

12. A method for selecting a spinach plant resistant to Peronospora effusa, comprising
a) identifying by genetic analysis the presence of an allele of the alpha-WOLF gene encoding a protein which confers at least resistance to Peronospora effusa races Pe:3, Pfs:5, Pe:8, Pe:9, Pe:11, Pe:14 and Pe:16 and does not confer resistance to races Pe:2, Pe:4, Pe:6, Pe:7, Pe:10, Pe:13, Pe:15 and Pe:18;
b) selecting a plant that comprises the allele of the alpha-WOLF gene;
c) optionally further testing the selected plant for Peronospora effuse; and
d) optionally selecting the further tested plant that exhibits Peronospora effuse resistance
wherein:
the allele of the alpha-WOLF gene comprises:
i) a nucleotide sequence comprising a coding sequence having at least 97% sequence identity to SEQ ID NO: 10; or
ii) a nucleotide sequence encoding a protein which has an amino acid sequence having at least 93.5% sequence identity to SEQ ID NO: 11; or
iii) a nucleotide sequence encoding an LRR domain wherein the nucleotide sequence has at least 94.5% sequence identity to SEQ ID NO: 12; or
iv) a nucleotide sequence encoding an LRR domain, wherein the LRR domain comprises an amino acid sequence having at least 93% sequence similarity to SEQ ID NO: 13; and
the protein encoded by the allele of the alpha-WOLF gene comprises in its amino acid sequence the motif MAEIGYSVC (SEQ ID NO: 1) at its N-terminus, the motif KWMCLR (SEQ ID NO: 2).

13. A method for producing a spinach plant exhibiting resistance to Peronospora effusa races Pe:3, Pfs:5, Pe:8, Pe:9, Pe:11, Pe:14 and Pe:16 comprising the steps of:
a) crossing a first spinach parent plant with a second spinach parent plant to obtain an F1 population, wherein the first spinach parent plant is the spinach plant of claim 1;
b) optionally performing one or more rounds of selfing and/or crossing with a F1 spinach plant to obtain a further generation population;
c) selecting from the F1 population resulting from the cross of step a), or from the further generation population of step b), a spinach plant that comprises the allele of the alpha-WOLF gene or exhibits resistance to Peronospora effusa races Pe:3, Pfs:5, Pe:8, Pe:9, Pe:11, Pe:14 and Pe:16.

14. A method for producing a hybrid spinach seed resistant to Peronospora effusa, comprising the steps of crossing a first spinach parent plant with a second spinach parent plant and harvesting the resultant hybrid spinach seed, wherein the first spinach parent plant and/or the second spinach parent plant is the spinach plant of claim 1 and comprises the allele of the alpha-WOLF gene homozygously.

15. The hybrid spinach seed produced by the method of claim 14.

16. A hybrid spinach plant grown from the hybrid spinach seed of claim 15.

17. The spinach plant of claim 1, wherein the spinach plant is an agronomically elite spinach plant.

18. The spinach plant of claim 17 wherein the agronomically elite spinach plant is a variety or an inbred line.

19. A seed of, or from, or that produces the plant of claim 1 and comprises the allele of the alpha-WOLF gene.

20. The method of claim 9 wherein the protein encoded by the allele of the alpha-WOLF gene further comprises in its amino acid sequence the motif DQEDEGEDN (SEQ ID NO: 4), and the method further comprises testing the plant identified as comprising the allele of the alpha-WOLF gene for exhibiting resistance to Peronospora effuse.

21. The method of claim 12, wherein the protein encoded by the allele of the alpha-WOLF gene further comprises in its amino acid sequence the motif DQEDEGEDN (SEQ ID NO: 4), and the method further comprises the steps of:
c) further testing the selected plant for Peronospora effuse resistance; and
d) selecting the further tested plant that exhibits Peronospora effuse resistance.

22. A method for producing a spinach plant comprising an allele of an alpha-WOLF gene comprising the steps of:
(a) crossing a first spinach parent plant with a second spinach parent plant to obtain an F1 population;
(b) optionally performing one or more rounds of selfing and/or crossing with a F1 spinach plant to obtain a further generation population;
(c) selecting from the population resulting from the cross of step a), or from the further generation population of step b), a spinach plant that comprises the allele of the alpha-WOLF gene;
wherein said allele of the alpha-WOLF gene:
encodes a protein which confers resistance to at least Peronospora effusa races Pe:3, Pfs:5, Pe:8, Pe:9, Pe:11, Pe:14 and Pe:16 and does not confer resistance to races Pe:2, Pe:4, Pe:6, Pe:7, Pe:10, Pe:13, Pe:15 and Pe:18;
i) a nucleotide sequence comprising a coding sequence having at least 97% sequence identity to SEQ ID NO: 10; or
ii) a nucleotide sequence encoding a protein which has an amino acid sequence having at least 93.5% sequence identity to SEQ ID NO: 11; or
iii) a nucleotide sequence encoding an LRR domain wherein the nucleotide sequence has at least 94.5% sequence identity to SEQ ID NO: 12; or
iv) a nucleotide sequence encoding an LRR domain, wherein the LRR domain comprises an amino acid sequence having at least 93% sequence similarity to SEQ ID NO: 13; and
the protein encoded by the allele of the alpha-WOLF gene comprises in its amino acid sequence the motif MAEIGYSVC (SEQ ID NO: 1) at its N-terminus, the motif KWMCLR (SEQ ID NO: 2).

23. The method of claim 22, wherein the protein encoded by the allele of the alpha-WOLF gene further comprises in its amino acid sequence the motif DQEDEGEDN (SEQ ID NO: 4), and the method further comprises the steps of:
d) further testing the selected plant for Peronospora effuse resistance; and
e) selecting the further tested plant that exhibits Peronospora effuse resistance.


SEQUENCE LISTING (15 sequences)

SEQ ID NO: 1 - protein, 9 aa, Spinacia oleracea
  REGION 1..9: note = motif
MAEIGYSVC

SEQ ID NO: 2 - protein, 6 aa, Spinacia oleracea
  REGION 1..6: note = motif
KWMCLR

SEQ ID NO: 3 - protein, 8 aa, Spinacia oleracea
  REGION 1..8: note = motif
HVGCVVDR

SEQ ID NO: 4 - DNA, 20 bp, Spinacia oleracea
  misc_feature 1..20: note = Forward primer LRR domain Alpha
  source 1..20: mol_type = genomic DNA
acaagtggatgtgtcttagg

SEQ ID NO: 5 - DNA, 19 bp, Spinacia oleracea
  misc_feature 1..19: note = Reverse primer LRR domain Alpha
  source 1..19: mol_type = genomic DNA
ttcgccctcatcttcctgg

SEQ ID NO: 6 - DNA, 18 bp, Spinacia oleracea
  misc_feature 1..18: note = Forward primer LRR domain Beta
  source 1..18: mol_type = genomic DNA
tcacgtgggttgtgttgt

SEQ ID NO: 7 - DNA, 1597 bp, Spinacia oleracea
  misc_feature 1..1597: note = Amplicon of LRR domain of the beta-WOLF 0 allele (Viroflay)
  source 1..1597: mol_type = genomic DNA
tcacgtgggttgtgttgtcgatagagatccagaaatagtctttttatgtagcaataagattcgttcgtatattagcggtcgctgcataaagaatccggtggattcacaaatagacaactggatgtgccttagggtgttggacttgtcagattcatgtgttaaagatttgtctgattcaataggtaagctgctgcacttaaggtatcttaacctctcttctaatataaagttggagataatccctgatgcaattacaagactgcataacttgcagacactacttttagaagattgcagaagtttaaaggagttgccaaaagatttttgcaaattggtcaaactgaggcacttggaattacagggttgtcatgatttgattggtatgtcatttggaatggataagctaactagtcttagaatactaccaaacattgtggtgggtaggaaggaacaaagtgttgatgatgagctgaaagccctaaaaggcctcaccgagataaaaggctccattgatatcacaatctattcaaaatatagaagagttgaaggcatgaatggcacaggaggaggagctgggtatttgaagagcatgaaacatctcacgggggttaatattacatttgatgaaggtggatgtgttaaccctgaagctgtgtatttgaagagcatgaaacatctcacgagggttattattatatttgattataaaggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgtccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcaccataatcggaaatcacggaataaataaagtgaagagactgtctggaagaacagggttggagcatttcactctgttggaatcactcaaactttcagatatagaagaccaggaagatgagggcgaa

SEQ ID NO: 8 - protein, 532 aa, Spinacia oleracea
  REGION 1..532: note = Amino acid sequence encoded by amplicon of LRR domain Beta Wolf 0(Viroflay)
HVGCVVDRDPEIVFLCSNKIRSYISGRCIKNPVDSQIDNWMCLRVLDLSDSCVKDLSDSIGKLLHLRYLNLSSNIKLEIIPDAITRLHNLQTLLLEDCRSLKELPKDFCKLVKLRHLELQGCHDLIGMSFGMDKLTSLRILPNIVVGRKEQSVDDELKALKGLTEIKGSIDITIYSKYRRVEGMNGTGGGAGYLKSMKHLTGVNITFDEGGCVNPEAVYLKSMKHLTRVIIIFDYKGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLTIIGNHGINKVKRLSGRTGLEHFTLLESLKLSDIEDQEDEGE

SEQ ID NO: 9 - DNA, 1398 bp, Spinacia oleracea
  misc_feature 1..1398: note = Amplicon of LRR domain of the alpha-WOLF 23 allele
  source 1..1398: mol_type = genomic DNA
tggatgtgtcttagggtgttggacttgtcaaggtcggatgttaaaaatttgcctaattcaataggtaaattgttgcacttgaggtgtcttaacctgtcttataatgatctgttgatactccctgatgcaattacaagactgcataatttgcagacactgcttttaaaagattgcggaagtttaatggagttgccaaaagatttttgcaaattggtcaaactgaggcacttggaattacagggttgtcatgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgggggttaatattagatttgatgatagagaaggtgtatttgttaaccctgaagctgtgttggcaaccctagagccaccttcaaatatcaagaggttagaggtgtggcattacgatggtacaacaattccagtatggggaagagcagagattaattgggcaatctccctctcgcatcttgttgacatcgagctttggcgttgtagtaatttgcaggagatgccagtgctgagtaaactgcctcatttgaaatcactgaaacttgaagatttgaataacttagagtacatggagagtagaagcagcagcagtagcagtgacacagaagcagcaacaccagaattaccaacattcttcccttcccttgaaaaacttacactttggcgtctgaacaagttgaagagttttgggaacaggagatcgagtagttttccccgcctctctgaattggaaatctgggaatgcccagatctaacgtggtttcctccctgtccaagccttgaagagttgacattgaaagacaaccatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatctgggttatctcaaatcactgcccacaaattgtcttactcacctcgaccttacaataagtgattccaaggagggggagggtgaatgggaagttggggatgcatttcagaagtgtgtatcttctttgagaaagctcagcataatcggaaatcacggaataaataaagtgaagagactgtctggaagaacagggttggagcatttcactctgttggactcactcgaactttcaaatatagaagaccaggaagatgagggcgaa

SEQ ID NO: 10 - protein, 466 aa, Spinacia oleracea
  REGION 1..466: note = Amino acid sequence encoded by amplicon of LRR domain ofalpha-WOLF 23
WMCLRVLDLSRSDVKNLPNSIGKLLHLRCLNLSYNDLLILPDAITRLHNLQTLLLKDCGSLMELPKDFCKLVKLRHLELQGCHDLIGMPLGMDRLTSLRVLPFFVVGRKEQSVDDELKALKGLTEIKGSIRIRIHSKYRIVEGMNDTGGAGYLKSMKHLTGVNIRFDDREGVFVNPEAVLATLEPPSNIKRLEVWHYDGTTIPVWGRAEINWAISLSHLVDIELWRCSNLQEMPVLSKLPHLKSLKLEDLNNLEYMESRSSSSSSDTEAATPELPTFFPSLEKLTLWRLNKLKSFGNRRSSSFPRLSELEIWECPDLTWFPPCPSLEELTLKDNHEALQIIVKITTTRGKEEKEEDKNAGVGNSQDDDNVKLRKVEIDNLGYLKSLPTNCLTHLDLTISDSKEGEGEWEVGDAFQKCVSSLRKLSIIGNHGINKVKRLSGRTGLEHFTLLDSLELSNIEDQEDEGE

SEQ ID NO: 11 - DNA, 3456 bp, Spinacia oleracea
  misc_feature 1..3456: note = Coding sequence of the alpha-WOLF 23 allele
  source 1..3456: mol_type = genomic DNA
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgatgtatggaacgaggatcgtgagaagtggtttaaattggaagagttgttaatgttgggtcaagggggaagcaaggttgtagtgaccgcacgttcagagaagacagcaaatgtcatagggaaaagacatttttatacactggaatgtttgtcgccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataacaagattttgtcgatattgaagctcagttactacaatcttgcaaactctttgaagagttgttttagttattgtgcagtgtttcccaaggatcatgaaataaagaaggagatgttgattgaactttggatggcacaaggatatgttgtgccgttggatggaggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgctaatacaaagaacttgggtgataaaatccgtcatgtacattgtgatgtcaatagatatgcacaaagagtctctctgtgtagccataagattcgttcgtatattggtggtcaatgtgaaaaacgttgggtggatacactaatagacaagtggatgtgtcttagggtgttggacttgtcaaggtcggatgttaaaaatttgcctaattcaataggtaaattgttgcacttgaggtgtcttaacctgtcttataatgatctgttgatactccctgatgcaattacaagactgcataatttgcagacactgcttttaaaagattgcggaagtttaatggagttgccaaaagatttttgcaaattggtcaaactgaggcacttggaattacagggttgtcatgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgggggttaatattagatttgatgatagagaaggtgtatttgttaaccctgaagctgtgttggcaaccctagagccaccttcaaatatcaagaggttagaggtgtggcattacgatggtacaacaattccagtatggggaagagcagagattaattgggcaatctccctctcgcatcttgttgacatcgagctttggcgttgtagtaatttgcaggagatgccagtgctgagtaaactgcctcatttgaaatcactgaaacttgaagatttgaataacttagagtacatggagagtagaagcagcagcagtagcagtgacacagaagcagcaacaccagaattaccaacattcttcccttcccttgaaaaacttacactttggcgtctgaacaagttgaagagttttgggaacaggagatcgagtagttttccccgcctctctgaattggaaatctgggaatgcccagatctaacgtggtttcctccctgtccaagccttgaagagttgacattgaaagacaaccatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatctgggttatctcaaatcactgcccacaaattgtcttactcacctcgaccttacaataagtgattccaaggagggggagggtgaatgggaagttggggatgcatttcagaagtgtgtatcttctttgagaaagctcagcataatcggaaatcacggaataaataaagtgaagagactgtctggaagaacagggttggagcatttcactctgttggactcactcgaactttcaaatatagaagaccaggaagatgagggcgaagacaacatcatattctggaaatcctttcctcaaaacctccgcagtttggaaattgaagactctgacaaaatgacaagtttgcccatggggatgcagtacttaacctccctccaaaccctcgaactatcatattgtgatgaattgaattcccttccagaatggataagcagcttatcatctcttcaatacctgggcatattcaactgtccagccctgaaatcactaccagaagcaatgcggaacctcacctcccttcagacacttgggatatcggattgtccagacctagttaaaatatgcagaaaacccaacggcgaggactatcccaaaattcaatacatccccaaaattattatatggtag

SEQ ID NO: 12 - protein, 1151 aa, Spinacia oleracea
  REGION 1..1151: note = Amino acid sequence of the alpha-WOLF 23 allele
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNDNEACSFLTIVGAGGLGKTALAQLVFNDERVKIEFHDLRYWVCVSDQDGGQFDVKEILCKILEVVTKEKVDNSSALELVQSQFQEKLRGKKYFLVLDDVWNEDREKWFKLEELLMLGQGGSKVVVTARSEKTANVIGKRHFYTLECLSPDYSWSLFEMSAFQKGHEQENHDELVDIGKKIVEKCYNNPLAITVVGSLLYGEEISKWRSFEMSELAKIGNGDNKILSILKLSYYNLANSLKSCFSYCAVFPKDHEIKKEMLIELWMAQGYVVPLDGGQSIEDAAEEHFVILLRRCFFQDVVKDEYGDVDSVKIHDLMHDVAQEVGREEICIVNANTKNLGDKIRHVHCDVNRYAQRVSLCSHKIRSYIGGQCEKRWVDTLIDKWMCLRVLDLSRSDVKNLPNSIGKLLHLRCLNLSYNDLLILPDAITRLHNLQTLLLKDCGSLMELPKDFCKLVKLRHLELQGCHDLIGMPLGMDRLTSLRVLPFFVVGRKEQSVDDELKALKGLTEIKGSIRIRIHSKYRIVEGMNDTGGAGYLKSMKHLTGVNIRFDDREGVFVNPEAVLATLEPPSNIKRLEVWHYDGTTIPVWGRAEINWAISLSHLVDIELWRCSNLQEMPVLSKLPHLKSLKLEDLNNLEYMESRSSSSSSDTEAATPELPTFFPSLEKLTLWRLNKLKSFGNRRSSSFPRLSELEIWECPDLTWFPPCPSLEELTLKDNHEALQIIVKITTTRGKEEKEEDKNAGVGNSQDDDNVKLRKVEIDNLGYLKSLPTNCLTHLDLTISDSKEGEGEWEVGDAFQKCVSSLRKLSIIGNHGINKVKRLSGRTGLEHFTLLDSLELSNIEDQEDEGEDNIIFWKSFPQNLRSLEIEDSDKMTSLPMGMQYLTSLQTLELSYCDELNSLPEWISSLSSLQYLGIFNCPALKSLPEAMRNLTSLQTLGISDCPDLVKICRKPNGEDYPKIQYIPKIIIW

SEQ ID NO: 13 - protein, 9 aa, Spinacia oleracea
  REGION 1..9: note = motif
DQEDEGEDN

SEQ ID NO: 14 - DNA, 30 bp, Spinacia oleracea
  misc_feature 1..30: note = standard amplification sequence
  source 1..30: mol_type = genomic DNA
gcagtcgaacatgtagctgactcaggtcac

SEQ ID NO: 15 - DNA, 30 bp, Spinacia oleracea
  misc_feature 1..30: note = standard amplification sequence
  source 1..30: mol_type = genomic DNA
tggatcacttgtgcaagcatcacatcgtag